(19) United States
(12) Patent Application Publication
(10) Pub. No.: US 2018/0275114 A1
(43) Pub. Date: Sep. 27, 2018

(54) DETECTING TRACER BREAKTHROUGH FROM MULTIPLE WELLS COMMINGLED AT A GAS OIL SEPARATION PLANT

(71) Applicant: Saudi Arabian Oil Company, Dhahran (SA)

(72) Inventors: Dmitry Kosynkin, Dhahran (SA); Erika Shoemaker Ellis, Dhahran (SA); Mohammed Alaskar, Khobar (SA); Afnan Mashat, Dhahran (SA)

(73) Assignee: Saudi Arabian Oil Company, Dhahran (SA)

(21) Appl. No.: 15/927,288

(22) Filed: Mar. 21, 2018

Related U.S. Application Data
(60) Provisional application No. 62/475,685, filed on Mar. 23, 2017.

(51) Int. Cl. G01N 33/28; E21B 43/34; E21B 49/08; G01N 1/14; G01N 21/64; G01N 30/72

(52) U.S. Cl. CPC G01N 33/2882; E21B 43/34; E21B 49/086; G01N 2001/1025; G01N 21/64; G01N 30/7206; G01N 33/2823; G01N 1/14

(57) ABSTRACT

Methods and systems for determining tracer breakthrough from multiple wells commingled at a Gas Oil Separation Plant (GOSP) are provided. An example hydrocarbon reservoir monitoring method includes periodically sampling fluid carried by a water line of a Gas Oil Separation Plant (GOSP). The GOSP is configured to receive commingled well hydrocarbon fluids from multiple wells formed in multiple regions of a hydrocarbon reservoir, separate the well fluids into hydrocarbon components including water, and flow the water through the water line. The multiple regions are tagged with respective tracers. Each tracer is injected into a respective region of the hydrocarbon reservoir surrounding a respective well and can flow into the respective well in response to a well breakthrough. The method also includes analyzing each sampled fluid for one or more tracers of the multiple tracers and monitoring the multiple wells for fluid breakthrough based on results of analyzing each sampled fluid.

### CROSS-REFERENCE TO RELATED APPLICATION

This application claims priority under 35 USC § 119(e) to U.S. Provisional Patent Application Ser. No. 62/475,685, filed on Mar. 23, 2017, the entire content of which is hereby incorporated by reference.

### TECHNICAL FIELD

This specification relates to hydrocarbon producing well systems, particularly with tracer detection systems.

### BACKGROUND

Tracers are commonly used in the oil industry for tracking oil and water flow patterns through reservoirs. Tracers can be injected into a reservoir, and afterwards producing lines are sampled to determine concentrations of the tracers that are present at the time of their arrival. The concentrations data can be used to understand fluid flow patterns and to infer other properties of the reservoir, for example, pore volumes and flow characteristics. Data from multi-well tracer tests can provide valuable information regarding well connectivity, sweep efficiency, and identification of geologic anomalies including fracture and super K zones.

Presently, tracer breakthrough and monitoring from an individual well is done manually by collecting samples on a daily or weekly basis, transporting fluid samples to a laboratory and purifying and measuring the fluid for tracer content. This requires daily or weekly operator intervention and a long turnover time for sample measurements.

### SUMMARY

The present specification describes methods and systems for determining tracer breakthrough from multiple wells simultaneously commingled into one common line at a Gas Oil Separation Plant (GOSP) with an automated, inline tracer detection system.

One aspect of the present specification features a well tracer detection system. The well tracer detection system includes a tracer detection system in fluid communication with a water line of a Gas Oil Separation Plant (GOSP) via a fluid sampling line fluidically connected to the water line. The GOSP is configured to receive commingled well hydrocarbon fluids from multiple wells. Each of the wells is in communication with a corresponding subterranean zone of a hydrocarbon reservoir. The corresponding subterranean zone is tagged with a respective tracer, and the respective tracer can flow into the well in response to a well breakthrough. The GOSP is also configured to separate the commingled well hydrocarbon fluids into hydrocarbon components including water, and flow the water through the water line. The tracer detection system receives a fluid sample from the fluid sampling line and analyzes the fluid sample to determine a presence of the tracers with which the subterranean zones corresponding to the multiple wells were tagged. The tracer detection system identifies a tracer in the fluid sample, and a well corresponding to a subterranean zone tagged with the identified tracer.

The tracer detection system can be configured to detect tracers in a parts per trillion (ppt) or parts per quadrillion (ppq) range. A volume of the fluid sample can be substantially 50 milliliters or less. The tracer detection system can include at least one of a laser-driven fluorescent spectrometer or a gas chromatography mass spectrometer. Each tracer can include at least one of an optically-tagged nanoparticle tracer or a mass-tagged nanoparticle tracer.

In some implementations, the system includes a fluid valve fluidically connecting the water line and the fluid sampling line. The fluid valve is configured to actuate to transfer the fluid sample from the water line to the fluid sampling line. The system can further include a filter positioned between the fluid sampling line and the tracer detection system. The filter is configured to remove contaminants in the fluid sample. The contaminants exclude a tracer. The system can further include a pump configured to flow the fluid sample from the fluid sampling line to the tracer detection system. The system can include a transmitter configured to transmit an identity of the identified well to a control station.

In some implementations, the system includes a computer system including one or more processors and a computer-readable medium. The computer-readable medium stores multiple first identifiers identifying the corresponding multiple wells and multiple second identifiers identifying the tracers with which the subterranean zones corresponding to the multiple wells are tagged. The computer-readable medium also stores computer instructions executable by the one or more processors to perform operations to identify the well tagged with the identified tracer. The computer system can identify a second identifier identifying the tracer identified by the tracer detection system from among the multiple second identifiers, and identify a first identifier identifying the well corresponding to the subterranzean zone tagged with the identified tracer from among the multiple first identifiers.

Another aspect of the present specfiication features a method of identifying a well breakthrough. A fluid sample is flowed from a water line of a Gas Oil Separation Plant (GOSP) to a tracer detection system. The GOSP is configured to receive commingled well hydrocarbon fluids from multiple wells. Each of the wells is in communication with a corresponding subterranean zone of a hydrocarbon reservoir. The corresponding subterranean zone is tagged with a respective tracer, and the respective tracer can flow into the well in response to a well breakthrough. The GOSP is also configured to separate the commingled well hydrocarbon fluids into hydrocarbon components including water and flow the water through the water line. The fluid sample is analyzed for a presence of the tracers in the fluid sample. A subterranean zone is identified from the subterranean zones tagged with the tracers. A well corresponding to the identified subterranean zone is identified.

The fluid sample can be pumped to the tracer detection system by using a pump. The fluid sample can be also flowed to the tracer detection system by gravitaty. In some cases, an identity of the identified well is received and a flow of the identified tracer into the identified well at the identified well is monitored in the hydrocarbon reservoir.

In some implementations, to determine the tracer in the fluid sample, the tracer is detected by performing at least one of laser-driven fluorescent spectroscopy or gas chromatography mass spectroscopy on the fluid sample. A concentration of the tracer in the fluid sample can be in the parts per trillion (ppt) or parts per quadrillion (ppq) range. Each tracer can include at least one of an optically-tagged nanoparticle tracer or a mass-tagged nanoparticle tracer.

To identify the well tagged with the determined tracer , a first computer-stored identifier that identifies the tracer and a second computer-stored identifier that is mapped to the identified first computer-stored identifier can be identified. The second computer-stored identifier identifies the well. In some cases, multiple first identifiers identifying the corresponding tracers and multiple second identifiers identifying the corresponding multiple wells are stored in a computer-readable storage medium. The multiple first identifiers includes the first computer-stored identifier, and the multiple second identifiers includes the second computer-stored identifier. One or more processors is operatively coupled to the computer-readable storage medium and configured to search the multiple first identifiers and the multiple second identifiers for a first identifier that identifies the tracer and a second identifier mapped to the first identifier.

A fluid valve can be actuated to fluidically connect the water line and a fluid sampling line to transfer the fluid sample from the water line to the fluid sampling line that is fluidically connected to the tracer detection system. Contaminants from the fluid sample obtained in the fluid sampling line can be filtered before flowing the fluid sample to the tracer detection system. The contaminants can exclude a tracer.

A further aspect of the present specification features a hydrocarbon reservoir monitoring method. Sampling fluid carried by a water line of a Gas Oil Separation Plant (GOSP) is periodically sampled. The GOSP is configured to receive commingled well hydrocarbon fluids from multiple wells formed in multiple regions of a hydrocarbon reservoir, separate the commingled well hydrocarbon fluids into hydrocarbon components including water, and flow the water through the water line. The multiple regions are tagged with multiple tracers, and each tracer is injected into a respective region of the hydrocarbon reservoir surrounding a respective well. Each tracer can flow from the respective region into the respective well in response to a well breakthrough. Each sampled fluid can be analyzed for one or more tracers of the multiple tracers and monitoring the multiple wells for fluid breakthrough based on results of analyzing each sampled fluid.

The fluid can be sampled periodically, for example, once or twice in a day. In response to analyzing a fluid sample, a presence of a tracer can be determined in the fluid sample, and a region of the hydrocarbon reservoir surrounding a well into which the tracer was injected can be identified. The well for fluid breakthrough can be then monitored.

Implementations of the present specification provide methods of determining tracer breakthrough from multiple wells simultaneously through an automated, inline tracer detection system that can be safely installed at a produced water test line from a Gas Oil Separation Plant (GOSP). By the methods, issues of high pressure, sour gas, separation procedures, return lines, and electrical supply can be minimized or eliminated.

The automated, inline detection system can be configured to trigger a flag or alert when one of the commingled wells associated with different respective tracers shows a tracer breakthrough. Once one or more tracers are detected by the detection system at the GOSP, respective individual wells feeding the commingled line can be correlated to the tracers measured by the detection system, and data can be transmitted to a base station in real time as part of the entire automated process. Once the individual wells showing breakthrough are identified, the identified wells can be tested directly at the well heads using conventional tracer monitoring methods and systems (for example, portable or handheld) to determine tracer concentration and progress.

The automated, inline detection system can be installed at the produced water stream test line downstream of the separator (after separating oil and gas from a main stream), thereby presenting minimal or no risk and hazardous high pressure sampling situations. The automated, inline detection system can include an actuated valve configured to release small quantities of the produced water into the detection system, an inline membrane separator and filter to remove any contaminants or emulsions, and a tracer detection system, based on either fluorescent mass spectrometers or gas chromatography mass spectrometers (GCMS), that are capable of detecting different tracers in a part per trillion (ppt) or part per quadrillion (ppq) level. The automated, inline detection system can also include a data acquisition module that flags any positive tracer detection and cross correlates the tracer to the appropriate well and a wireless data transfer device, for example, a transmitter, that sends this data in real time to a base control station. If no tracer is detected from the detection system, the detection system simply continues to monitor the flow stream. If there are any positive tracer flags signaling a breakthrough from one or more of the commingled wells, the data is automatically correlated to a tracer/well computer database to identify the well or wells showing breakthrough and the data transferred in real time to the base control station. When the transmitted data is reviewed and tracer breakthrough from specific wells are identified, conventional well head tracer monitoring can be deployed.

The technology can avoid time consuming procedure of monitoring every well injected with tracers on a daily or weekly basis to detect tracer breakthrough individually and manually. Instead, the technology enables to automatically determine tracer breakthrough from multiple commingled wells without expensive, high risk modifications to every well head for tracer detection. This can save considerable amount of man power and hours and potentially allow for tracers to be deployed at a reservoir or field scale at an efficient and economical way. In addition, enabling field-wide implementation of tracers studies can provide important information about fluid movements within the reservoir, which can result in optimized water injection schemes and informative infill drilling, and improved reservoir management strategies and ultimately increasing oil recovery.

The details of one or more implementations of the subject matter of this specification are set forth in the accompanying drawings and associated description. Other features, aspects, and advantages of the subject matter will become apparent from the description, the drawings, and the claims.

### DESCRIPTION OF DRAWINGS

FIG. 1A is a schematic diagram illustrating an example of a hydrocarbon producing well system.

FIG. 1B is a schematic diagram illustrating an example of a tracer detection system.

FIG. 2 is a schematic diagram illustrating an example of transmission between hydrocarbon producing well systems and a base station.

FIG. 3A is a flowchart of an example process of determining tracer breakthrough from multiple wells commingled at a GOSP.

FIG. 3B is a flowchart of an example process of determining a well tagged with an identified tracer.

FIG. 4 is a block diagram of an example of a computer system.

### DETAILED DESCRIPTION

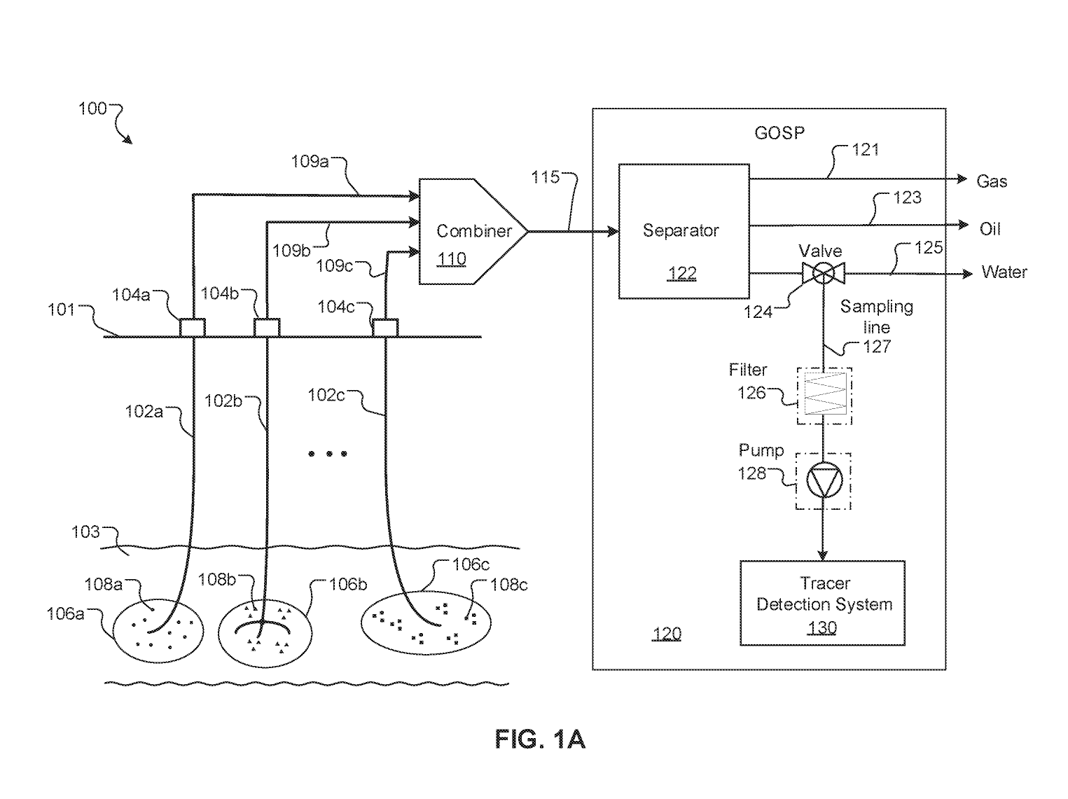

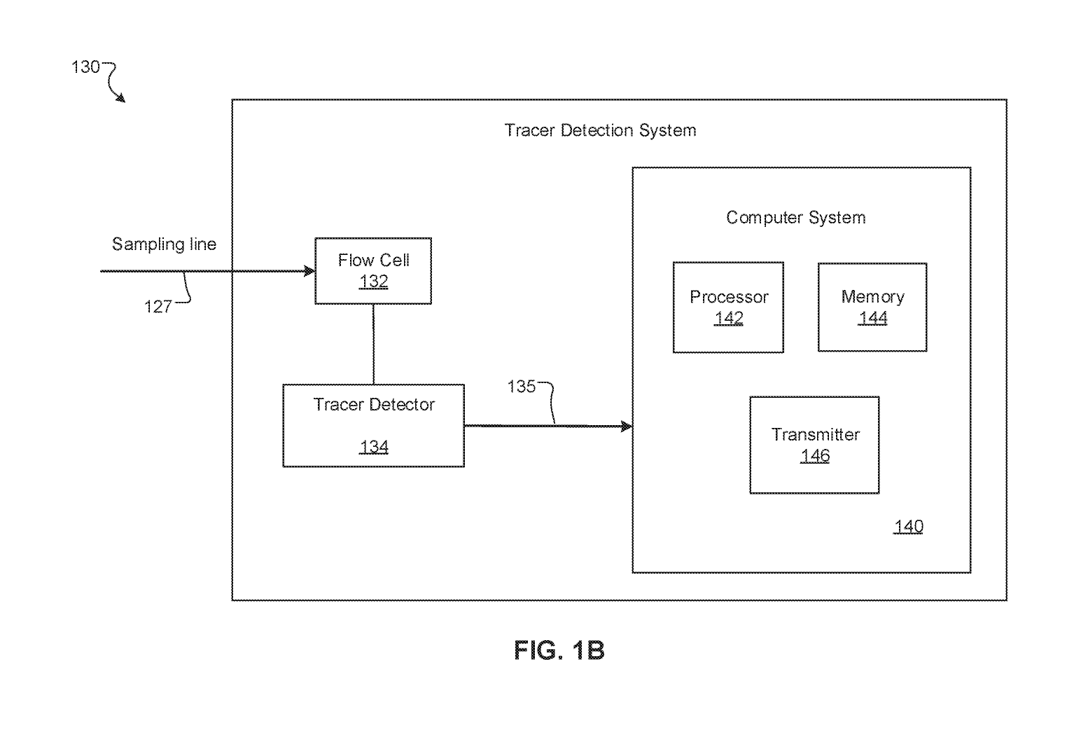

FIG. 1A is a schematic diagram illustrating an example of a hydrocarbon producing well system 100 provided by the present specification. The well system 100 can be arranged at a well site. The well system 100 includes multiple wells 102a, 102b, 102c that are separate from each other but located within the same well site, that is, formed in the same hydrocarbon reservoir from which hydrocarbons are being extracted.

Each well 102a, 102b, or 102c extends from a respective well head 104a, 104b, or 104c at a terranean surface (or the Earth's surface) 101 through a respective subterranean zone of interest 106a, 106b, or 106c in a hydrocarbon reservoir 103. For example, the well 102a extends from the well head 104a at the terranenan surface 101 through the subterranean zone 106a in the hydrocarbon reservoir 103. The well 102a, 102b, or 102c can be any suitable type of well, for example, a well including a single wellbore like the well 102a or 102c, or a well including multiple wellbores like the well 102b. The well 102a, 102b, or 102c is configured to produce hydrocarbon components, for example, gas, oil, water, or any suitable combinations, from the subterranean zone 106a, 106b, or 106c, respectively. The subterranean zone 106a, 106b, or 106c surrounds the well 102a, 102b, or 102c, respectively. Each well 102a, 102b, or 102c is in communication, for example, fluidically, with a corresponding subterranean zone 106a, 106b, or 106c.

Individual tracers 108a, 108b, 108c are injected into the respective subterranean zones 106a, 106b, 106c. In response to a well breakthrough of the well 102a, 102b, or 102c, the tracers 108a, 108b, or 108c can flow into the respective well 102a, 102b, or 102c, respectively. The tracers 108a, 108b, 108c can be different from each other, so that individual tracers in each well can be used to uniquely identify a respective well breakthrough of the well. That is, each well 102a, 102b, or 102c corresponds to a subterranean zone 106a, 106b, 106c tagged with respective tracers 108a, 108b, or 108c. Each tracer 108a, 108b, or 108c is associated with (or corresponds to) a respective well 102a, 102b, or 102c.

In some cases, the tracers are optically-tagged nanoparticle tracers and can be detected by optical methods, for example, laser-driven fluorescent spectroscopy or Raman spectroscopy. Nanoparticle tracers with different sizes (lengths, diameters) or shapes or both, may have different fluorescence spectrums (for example, emission wavelengths), Raman spectrums, or resonance spectrums. In some cases, the tracers are mass-tagged nanoparticle tracers and can be detected by mass spectrometers, for example, gas (or liquid) chromatography mass spectrometers (GCMS or LCMS). The tracers can also be any other suitable nanoparticle tracers. For example, this detection scheme could be equally applicable for molecule base chemical tracers, DNA strands tags, and bio-based tracers.

Hydrocarbon liquids produced from the multiple wells 102a, 102b, 102c can be input through respective lines 109a, 109b, 109c to a combiner 110 that is configured to combine the hydrocarbon liquids to be commingled well hydrocarbon fluids. The commingled well hydrocarbon fluids are further sent from the combiner 110 through a common line 115 to a separator 122 of a Gas Oil Separation Plant (GOSP) 120. The separator 122 can be a residual oil/water separator or an oil/gas/water separator. As illustrated in FIG. 1A, the separator 122 is configured to separate the commingled well hydrocarbon fluids received from the combiner 110 into separated lines 121, 123, 125 to get separated hydrocarbon components comprising gas, oil, and water.

A fluid sampling line 127 is configured to fluidically couple to the water line 125 of the GOSP 120 to obtain a fluid sample from the water flowing through the water line 125. In some implementations, a fluidic valve 124 fluidically connects the water line 125 and the fluid sampling line 127 and is configured to actuate to transfer the fluid sample from the water line 125 to the fluid sampling line 127. The fluid sample can be small quantities of produced water. For example, a volume of the fluid sample can be substantially 50 milliliter (mL) or less. The fluidic valve 124 can be actuated periodically, for example, once or twice per day, or in response to a request.

The GOSP 120 includes a tracer detection system 130 configured to identify one or more tracers in the fluid sample. The tracer detection system 130 is fluidically coupled to the fluid sampling line 127 and configured to receive the fluid sample from the fluid sampling line 127, analyze the fluid sample to determine a presence of the multiple tracers 108a, 108b, 108c with which the subterranean zones 106a, 106b, 106c corresponding to multiple wells 102a, 102b, 102c were tagged, identify, in response to analyzing the fluid sample, a tracer in the fluid sample, and identify, in response to identifying the tracer in the fluid sample, a well corresponding to a subterranean zone tagged with the identified tracer. The tracer detection system 130 is configured to detect tracers in a ppt or ppq range. The tracer detection system 130 can be an automated, inline measurement system installed downstream of the separator 122 in the GOSP 120. As discussed further in FIG. 1B later, if the tracers are nanoparticles with fluorescence materials, the tracer detection system 130 can detect an optical spectrum (or an emission spectrum) of the fluid sample excited by a white LED light. It can be determined the presence of the multiple tracers based on the optical spectrum and further determined individual tracers based on respective resonance wavelengths (or emission wavelengths) in the optical spectrum. If the tracers are mass-tagged nanoparticle tracers, the tracer detection system 130 can detect a mass spectrum of the fluid sample. It can be determined the presence of the multiple tracers based on the mass spectrum and further determined individual tracers based on respective signature peaks in the mass spectrum.

In some implementations, the tracer detection system 130 is configured to detect a particular tracer tagged to a particular subterranean zone corresponding to a particular well. For example, if the particular tracer includes nanoparticles with a fluorophore material, in a tracer detection test, the tracer detection system 130 can use a laser source with a wavelength within an absorption range of the fluorescence material to illuminate the fluid sample and to detect an emission spectrum of the fluidic sample. If the emission wavelength in the detected emission spectrum matches with the emission wavelength for the fluorophore material, it can be determined that the fluid sample includes the particular tracer. In some cases, the tracer detection system 130 runs different tracer detection tests on the fluid sample for identifying different tracers, for example, in parallel or in series.

In some implementations, the GOSP 120 includes a filter 126 positioned between the fluid sampling line 127 and the tracer detection system 130. The filter 126 is configured to remove contaminants or emulsions in the fluid sample. The contaminants or emulsions exclude tracers. The filter 126 can be an inline filter cartridge and include one or more water purification materials.

In some implementations, the fluidic sample or the filtered fluidic sample from the filter 126 is fed to the tracer detection system 130 gravitationally. In some implementations, the GOSP 120 includes a pump 128 configured to flow the fluid sample from the fluid sampling line 127 to the tracer detection system 130. The pump 128 can be arranged after (or before) the filter 126 between the fluidic sampling line 127 (or the fluidic valve 124) and the tracer detection system 130. The pump 128 can be a small capillary pump.

FIG. 1B is a schematic diagram illustrating an example of the tracer detection system 130. The tracer detection system 130 includes a flow cell 132 configured to receive the fluid sample from the fluid sampling line 127, for example, through the filter 126 or the pump 128 or both. The flow cell 132 can be a container, for example, a cuvette, positioned on a holder. The tracer detection system 130 includes a tracer detector 134 configured to detect any tracers in the fluid sample. The tracer detector 134 can be configured to detect a number of different tracers in a ppt or ppq level. The number can be more than 10, 20, 50, or 100.

As noted earlier, in some examples, the tracers are optically-tagged nanoparticle tracers, for example, nanoparticles with fluorescent materials, quantum dots, or metal nanoparticles. The tracers can emit optical signals when excited by light, and the optical signals can be fluorescent signals or Raman scattering signals. Different tracers can have different resonance wavelengths or signature peaks. The tracer detection system 130 can include a light source for fluorescent or Raman illumination, for example, a light-emitted-diode (LED) lamp, to inject light on the flow cell 132. The tracer detector 134 can be a spectrometer configured to measure an optical spectrum of the emitted optical signals. Based on resonance wavelengths or signature peaks in the optical spectrum, one or more tracer types can be identified. For example, the tracers 108a, 108b, and 108c can be nanoparticles with different fluorophores with resonance wavelengths of 450 nanometer (nm), 530 nm, 630 nm, respectively. A white LED light can be used to illuminate the fluid sample. If the optical spectrum (or the emission spectrum) measured by the tracer detector 134 includes a resonance wavelength of 530 nm and a resonance wavelength of 630 nm, it can be determined that the fluid sample includes the tracers 108b and the tracers 108c.

In some examples, the tracers are mass-tagged nanoparticle tracers. The tracer detector 134 can be a mass spectrometer, for example, a gas (or liquid) chromatography mass spectrometers (GCMS or LCMS), that can uniquely detect the mass-tagged nanoparticle tracers based on their mass spectrums. For example, when a fluid sample is tested by the mass spectrometer, the mass spectrum includes signature peaks of the tracers 108a and 108b. Based on the signature peaks, it can be determined the presence of tracers and further determined that the fluid sample includes the tracers 108a and the tracers 108b.

Data output from the tracer detector 134, for example, fluorescence or Raman spectrums, mass spectrums of the fluid sample, can be transmitted to a computer system 140 via a connection 135. The connection 135 can be a wired line or a wireless connection. The tracer detector 134 can continuously monitor the fluid sample to detect tracer breakthrough without human intervention. The data can be continuously transmitted to the computer system 140.

The computer system 140 is configured to analyze the data to determine a presence of the multiple tracers 108a, 108b, 108c with which the subterranean zones 106a, 106b, 106c corresponding to the multiple wells 102a, 102b, 102c were tagged. As discussed earlier, this determination can be based on the resonance wavelengths (or signature peaks) in the fluorescence spectrums (or mass spectrums). Based on the resonance wavelengths or signature peaks, the computer system 140 can further identify one or more tracers in the fluid sample. If there is a tracer identified, it indicates that the tracer flows into a well in response to a well breakthrough of the well (or called a tracer breakthrough).

In some implementations, the computer system 140 includes one or more processors 142 and a memory (for example, a computer-readable medium) 144. The memory 144 stores multiple first identifiers identifying the corresponding multiple wells and multiple second identifiers identifying multiple tracers with which the subterranean zones corresponding to the multiple wells are tagged. That is, each first identifier identifying a well is associated with (or mapped to) a respective second identifier identifying a tracer with which a subterranean zone corresponding to the well is tagged. The memory stores a database including the associations (or mappings) of the first identifiers with the second identifiers.

The memory 144 can also store computer instructions executable by the one or more processors 142 to perform operations to identify the well corresponding to the subterranean zone tagged with the identified tracer. The computer system 140 can be configured to identify, from among the multiple second identifiers, a second identifier identifying the tracer identified and then identify, from among the multiple first identifiers, a first identifier identifying the well corresponding to the subterranean zone tagged with the identified tracer, based on an association between the first identifier and the second identifier. In other words, the computer system 140 can identify a second computer-stored identifier that identifies the tracer and then identify a first computer-stored identifier that is associated with (or mapped to) the identified second computer-stored identifier. The first computer-stored identifier identifies the well.

The computer system 140 can flag any positive tracer detection and cross-correlate the tracer to an associated well. The computer system 140 can determine whether there are any positive tracer flags signaling a breakthrough from one or more of the commingled wells 102a, 102b, 102c. If no tracer is detected, the tracer detection system 130 continues to sample and monitor for any breakthrough. If tracer(s) breakthrough is detected, the corresponding well(s) is identified by referencing the stored computer database in the memory 144.

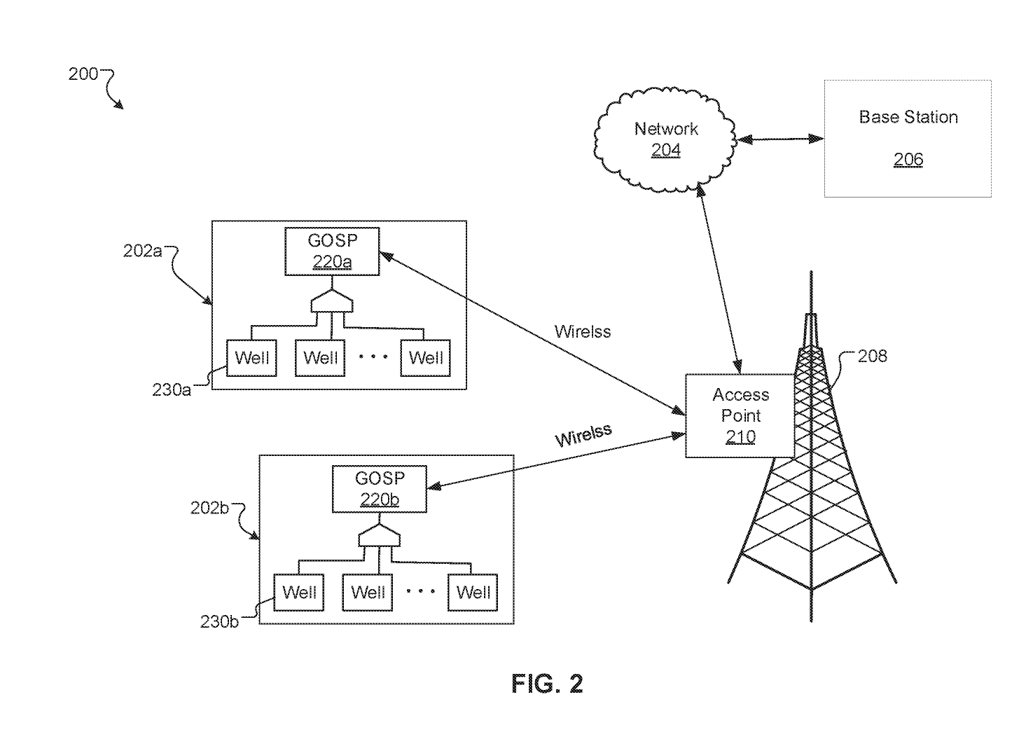

In some implementations, the computer system 140 includes a transmitter 146 configured to transmit, for example, wirelessly, an identity of the identified well to a control station, as discussed in further details in FIG. 2. The transmitter 146 can also transmit the measured data of the tracer detector 134, for example, the optical spectrums or mass spectrums, to the control station. At the control station, the transmitted data can be reviewed and tracer breakthrough from specific wells can be identified, and conventional well head tracer monitoring systems can begin as per standard field operations. The control station can also store a database associating tracers to wells. In some examples, the control station can identify a tracer based on the measured data and then identify a well based on the identified tracer and the database. In some examples, the control station can receive an identify of an identified well identified by an identified tracer and send a request or command to monitor, at the identified well in the hydrocarbon reservoir, a flow of the identified tracer into the identified well.

In some examples, the computer system 140 is included in the tracer detection system 130, as illustrated in FIG. 1B. In some other examples, the computer system 140 is externally coupled to the tracer detection system 130. The computer system 140 can be in the GOSP 120 or external to the GOSP 120. In some examples, the transmitter 146 is included in the computer system 140, as illustrated in FIG. 1B. In some other examples, the transmitter 146 is externally coupled to the computer system 140 but included in the tracer detection system 130 or in the GOSP 120.

FIG. 2 is a schematic diagram illustrating an example 200 of transmission between hydrocarbon producing well systems 202a, 202b and a base station 206. The well systems 202a, 202b can be at different well sites separate from each other. The well system 202a or 202b can be the well system 100 of FIG. 1A. Although two well systems are depicted, it is appreciated that the base station 206 can communicate with a number of well systems. In such a way, the base station 206 can remotely monitor (and control) numerous well systems at numerous well sites.

Each well system 202a or 202b can include a GOSP system 220a or 220b, respectively, configured to receive commingled well hydrocarbon fluids from multiple wells 230a or 230b, respectively. The GOSP system 220a or 220b can be the GOSP 120 of FIG. 1A. The GOSP system 220a or 220b can periodically sample fluid carried by a water line of the GOSP 220a or 220b to analyze for one or more of multiple tracers tagged to multiple subterranean zones corresponding to the multiple wells and monitor the multiple wells for fluid breakthrough based on results of analyzing each sampled fluid. The GOSP system 220a or 220b each can include a transmitter, for example, the transmitter 146 of FIG. 1B, and transmit data, for example, measured data or identified wells or both, to the base station 206, for example, wirelessly.

In some implementations, the GOSP system 220a or 220b wirelessly transmits data to the base station 206 through a network 204. The network 204 can include a large computer network, such as a local area network (LAN), wide area network (WAN), the Internet, a cellular network, a satellite network, a mesh network, one or more wireless access points, or a combination thereof connecting any number of mobile clients, fixed clients, and servers.

In some implementations, the data is transmitted from each GOSP system 220a or 220b through an access point 210 that can be mounted on a tower 208. The tower 208 can include an existing telecommunications tower. In some examples, data can be transmitted between the access point 210 and the GOSP systems 220a, 220b based on a local network, for example, a low power data network, a cellular network, a satellite communication network, or any combination thereof In some examples, the access point 210 provides a radial coverage that enables the access point 210 to communicate with numerous well systems, such as the well system 202a or 202b. In some examples, the access point 210 further communicates with the network 204 using cellular, satellite, mesh, point-to-point, point-to-multipoint radios, terrestrial or wired communication, or any combination thereof.

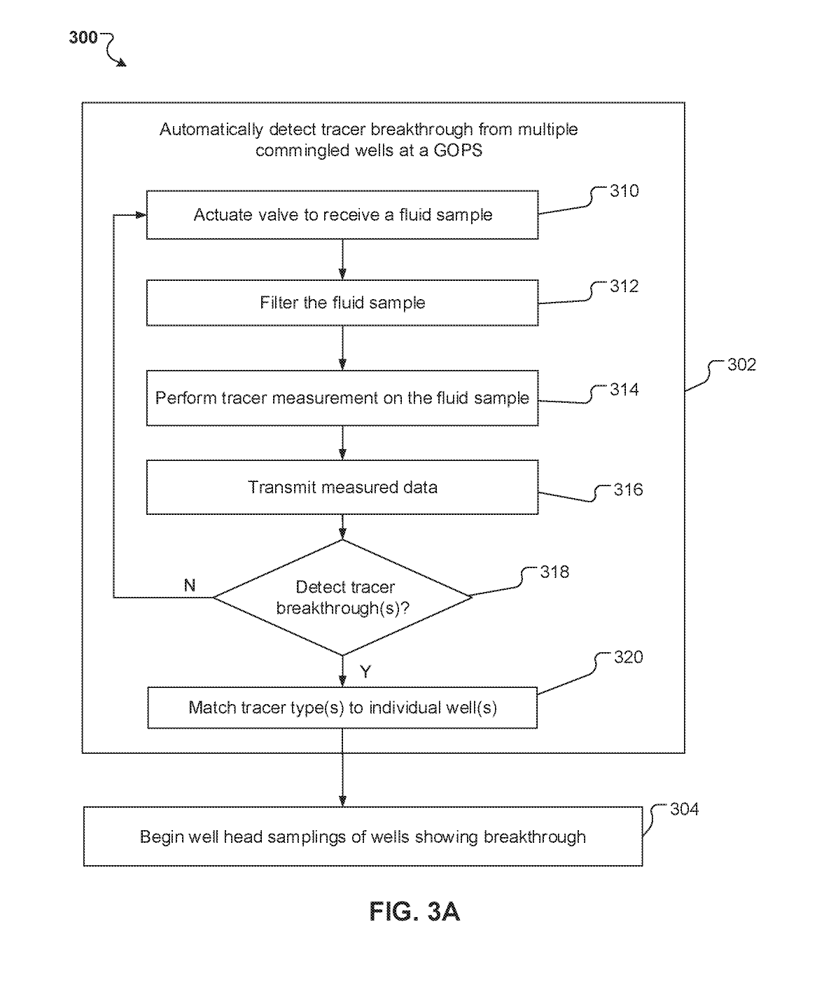

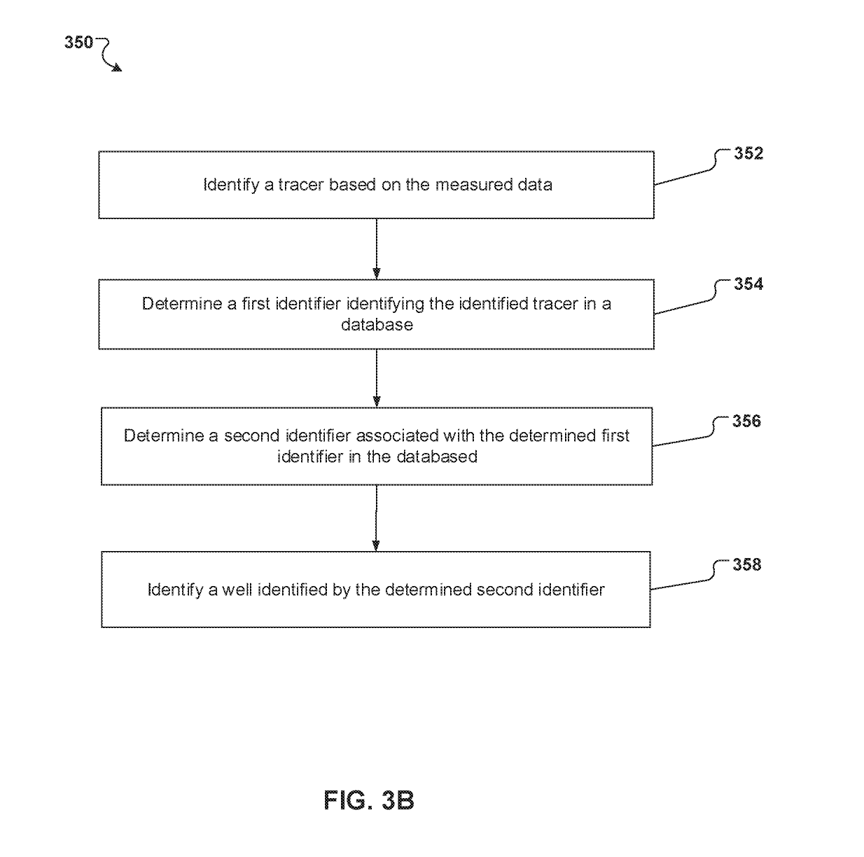

FIG. 3A is a flowchart of an example process 300 of determining tracer breakthrough from multiple wells commingled at a GOSP. The GOSP can be the GOSP 120 of FIG. 1A. The GOSP is configured to receive commingled well hydrocarbon fluids from the multiple wells formed in multiple regions of a hydrocarbon reservoir and separate, for example, by the separator 122 of FIG, 1A, the well fluids into hydrocarbon components including water. The GOSP is configured to flow the water through a water line and sample the produced water for detection.

Tracer breakthrough is detected from the multiple commingled wells at the GOSP (302). As noted earlier, each well corresponds to a region of the hydrocarbon reservoir surrounding with the well. A respective tracer is injected into the region and flows into the well in response to a well breakthrough. That is, each well is associated with a tracer tagged to a region corresponding to the well.

The detection 302 can be automatically performed at the GOSP. A valve, for example, the valve 124 of FIG. 1A, is actuated to receive a fluid sample (310) from the water line, for example, periodically such as once or twice per day. The sampled fluid can have a volume of substantially 50 mL or less. The fluid sample is filtered (312), for example, by the filter 126 of FIG. 1A. The filtered sample can be by gravity or pumped, for example, by the pump 128 of FIG. 1B, to a tracer detection system of the GOSP, for example, the tracer detection system 130 of FIGS. 1A-1B.

Tracer measurement is performed on the fluid sample (314) by the tracer detection system. The tracer measurement can be based on laser-driven fluorescent or Raman spectroscopy or mass spectrometer such as GCMS on the fluid sample. Each tracer can be at least one of an optically-tagged nanoparticle tracer or a mass-tagged nanoparticle tracer. The concentration of the tracer in the fluid sample can be in the ppt or ppq range.

The measured data is transmitted (316), for example, from the tracer detection system to a computer system, for example, the computer system 140 of FIG. 1B. The computer system determines whether there is any tracer breakthrough (318), for example, a tracer flowing into a well in response to a well breakthrough of the well. If the computer system determines that there is no tracer detected in the fluid sample, the process 300 goes back to step 310 for continuously sampling the fluid from the water line of the GOSP. If the computer system determines that there are one or more tracers detected in the fluid sample, the computer system matches the detected tracers to corresponding wells (320).

FIG. 3B is a flowchart of an example process 350 of determining a well associated with an identified tracer. The process 350 can be performed by the computer system and implemented as the step 320.

A tracer is identified based on measured data (352). The measured data can be from the tracer detection system. A first identifier identifying the identified tracer is determined in a database (354). The database can be stored in the computer system and associates or maps different tracers to respective wells corresponding to respective regions tagged with the tracers. A second identifier associated with the first identifier in the database is determined (356), for example, based on the association stored in the database. Then a well identified by the determined second identifier is determined (358).

Referring back to FIG. 3A, after the well(s) associated with the identified tracers in the fluid sample is (or are) identified, data including the identified well(s) or the measured data or both can be transmitted, for example, wirelessly, to a base station. In some cases, the data can be transmitted to the base station in real time as part of the entire automated process 302. That is, as soon as the tracer detection system obtains the data, the tracer detection system transmits the data to the base station. The base station can analyze the identified well(s) or the measured data or both and start a well head tracer monitoring system, for example, per standard field operations. Well head sampling of wells showing breakthrough is begun, for example, manually (304). In some implementations, steps 318 and 320 can be performed by the based station based on the measured data and a stored database associating tracers to wells.

This process 300 eliminates the need for constant manual testing of all injected wells daily for signs of tracer breakthrough and does not require expensive, high risk modifications to every well head for tracer detection. In this process 300, several tracers can be monitored by a continuous periodic sampling system safely (for example, an ongoing sampling program is running where samples are taking periodically), and manual testing at the well head can be delayed until tracer breakthrough is first detected at the GOSP. This process 300 can allow for field wide monitoring systems.

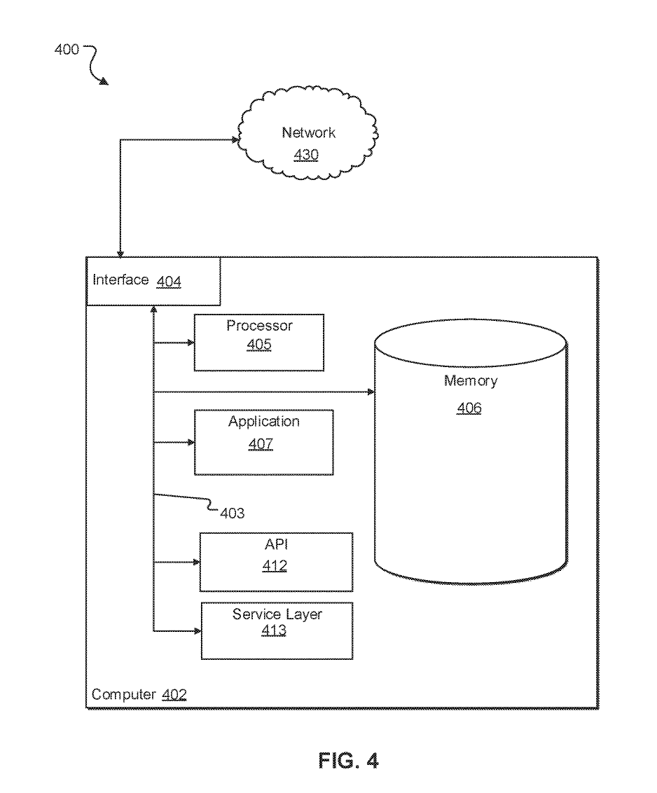

FIG. 4 is a block diagram 400 of an exemplary computer 402 used for determining tracer breakthrough from multiple wells commingled at a GOSP according to an implementation. The computer 402 can be the computer system 140 of FIG. 1B. The illustrated computer 402 is intended to encompass any computing device such as a server, desktop computer, laptop or notebook computer, wireless data port, smart phone, personal data assistant (PDA), tablet computing device, or one or more processors within these devices, including both physical and virtual instances of the computing device. Additionally, the computer 402 may comprise a computer that includes an input device, such as a keypad, keyboard, touch screen, or other device that can accept user information, and an output device that conveys information associated with the operation of the computer 402, including digital data, visual and audio information, or a graphical user interface (GUI).

The computer 402 can serve as a client, network component, a server, a database or other persistency, or a component of a computer system for determining tracer breakthrough from multiple wells commingled at a GOSP. The illustrated computer 402 is communicably coupled with a network 430. The network 430 can be the network 204 of FIG. 2. In some implementations, one or more components of the computer 402 may be configured to operate within a cloud-computing-based, local, global, or other environment.

At a high level, the computer 402 is an electronic computing device operable to receive, transmit, process, store, or manage data and information associated with determining tracer breakthrough from multiple wells commingled at a GOSP. According to some implementations, the computer 402 may also include or be communicably coupled with an application server, e-mail server, web server, caching server, streaming data server, business intelligence (BI) server, or other server.

The computer 402 can receive requests over network 430 from a client application (for example, executing on another computer 402) and respond to the received requests by processing the said requests in an appropriate software application. In addition, requests may also be sent to the computer 402 from internal users (for example, from a command console or by other appropriate access method), external or third parties, other automated applications, as well as any other appropriate entities, individuals, systems, or computers.

Each of the components of the computer 402 can communicate using a system bus 403. In some implementations, any or all the components of the computer 402, both hardware and software, may interface with each other or the interface 404 over the system bus 403 using an application programming interface (API) 412 or a service layer 413. The API 412 may include specifications for routines, data structures, and object classes. The API 412 may be either computer language-independent or -dependent and refer to a complete interface, a single function, or even a set of APIs. The service layer 413 provides software services to the computer 402 and other components (whether or not illustrated) that are communicably coupled to the computer 402. The functionality of the computer 402 may be accessible for all service consumers using this service layer. Software services, such as those provided by the service layer 413, provide reusable, defined business functionalities through a defined interface. For example, the interface may be software written in any suitable language providing data in extensible markup language (XML) format or other suitable format. While illustrated as an integrated component of the computer 402, alternative implementations may illustrate the API 412 and the service layer 413 as stand-alone components in relation to other components of the computer 402 and other components (whether or not illustrated) that are communicably coupled to the computer 402. Moreover, any or all parts of the API 412 and the service layer 413 may be implemented as child or sub-modules of another software module, enterprise application, or hardware module without departing from the scope of this specification.

The computer 402 includes an interface 404. Although illustrated as a single interface 404 in FIG. 4, two or more interfaces 404 may be used according to particular needs, desires, or particular implementations of the computer 402 and functionality for determining tracer breakthrough from multiple wells commingled at a GOSP. The interface 404 is used by the computer 402 for communicating with other systems in a distributed environment that are connected to the network 430. Generally, the interface 404 comprises logic encoded in software and hardware in a suitable combination and operable to communicate with the network 430. More specifically, the interface 404 may comprise software supporting one or more communication protocols associated with communications such that the network 430 or interface's hardware is operable to communicate signals within and outside of the illustrated computer 402.

The computer 402 includes a processor 405. The processor 405 can be the processor 142 of FIG. 1B. Although illustrated as a single processor 405 in FIG. 4, two or more processors may be used according to particular needs, desires, or particular implementations of the computer 402. Generally, the processor 405 executes instructions and manipulates data to perform the operations of the computer 402. Specifically, the processor 405 executes the functionality for determining tracer breakthrough from multiple wells commingled at a GOSP.

The computer 402 also includes a memory 406 that holds data for the computer 402 and other components that can be connected to the network 430. The memory 406 can be the memory 144 of FIG. 1B. For example, memory 406 can be a database storing data consistent with this specification. Although illustrated as a single memory 406 in FIG. 4, two or more memories may be used according to particular needs, desires, or particular implementations of the computer 402 and functionality to determine tracer breakthrough from multiple wells commingled at a GOSP. While memory 406 is illustrated as an integral component of the computer 402, in alternative implementations, memory 406 can be external to the computer 402.

The application 407 is an algorithmic software engine providing functionality according to particular needs, desires, or particular implementations of the computer 402, particularly with respect to functionality required for determining tracer breakthrough from multiple wells commingled at a GOSP. For example, application 407 can serve as one or more components, modules, and applications described with respect to any of the figures. Further, although illustrated as a single application 407, the application 407 may be implemented as multiple applications 407 on the computer 402. In addition, although illustrated as integral to the computer 402, in alternative implementations, the application 407 can be external to the computer 402.

There may be any number of computers 402 associated with, or external to, a computer system containing computer 402, each computer 402 communicating over network 430. Further, the terms “client,” “user,” and other appropriate terminology may be used interchangeably as appropriate without departing from the scope of this specification. Moreover, this specification contemplates that many users may use one computer 402, or that one user may use multiple computers 402.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Implementations of the subject matter described in this specification can be implemented as one or more computer programs, such as, one or more modules of computer program instructions encoded on a tangible, non-transitory computer-storage medium for execution by, or to control the operation of, data processing apparatus. Alternatively or in addition, the program instructions can be encoded on an artificially generated propagated signal, such as, a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of one or more of them.

The terms “data processing apparatus,” “computer,” or “electronic computer device” (or equivalent as understood by one of ordinary skill in the art) refer to data processing hardware and encompass all kinds of apparatus, devices, and machines for processing data, including by way of example, a programmable processor, a computer, or multiple processors or computers. The apparatus can also be or further include special purpose logic circuitry, for example, a central processing unit (CPU), an FPGA (field programmable gate array), or an ASIC (application-specific integrated circuit). In some implementations, the data processing apparatus and special purpose logic circuitry may be hardware-based and software-based. The apparatus can optionally include code that creates an execution environment for computer programs, for example, code that constitutes processor firmware, a protocol stack, a database management system, an operating system, or a combination of one or more of them. The present specification contemplates the use of data processing apparatuses with or without conventional operating systems.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, for example, one or more scripts stored in a markup language document, in a single file dedicated to the program in question, or in multiple coordinated files, for example, files that store one or more modules, sub-programs, or portions of code. A computer program can be deployed to be executed on one computer or on multiple computers that are located at one site or distributed across multiple sites and interconnected by a communication network. While portions of the programs illustrated in the various figures are shown as individual modules that implement the various features and functionality through various objects, methods, or other processes, the programs may instead include a number of sub-modules, third-party services, components, libraries, and such, as appropriate. Conversely, the features and functionality of various components can be combined into single components as appropriate.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, such as, a CPU, an FPGA, or an ASIC.

Computers suitable for the execution of a computer program can be based on general or special purpose microprocessors, both. Generally, a CPU will receive instructions and data from a read-only memory (ROM) or a random access memory (RAM) or both. The essential elements of a computer are a CPU for performing or executing instructions and one or more memory devices for storing instructions and data. Generally, a computer will also include, or be operatively coupled to, receive data from or transfer data to, or both, one or more mass storage devices for storing data, for example, magnetic, magneto-optical disks, or optical disks. However, a computer need not have such devices. Moreover, a computer can be embedded in another device, for example, a mobile telephone, a personal digital assistant (PDA), a mobile audio or video player, a game console, a global positioning system (GPS) receiver, or a portable storage device, for example, a universal serial bus (USB) flash drive, to name just a few.

Computer-readable media (transitory or non-transitory, as appropriate) suitable for storing computer program instructions and data include all forms of non-volatile memory, media and memory devices, including by way of example semiconductor memory devices, for example, erasable programmable read-only memory (EPROM), electrically erasable programmable read-only memory (EEPROM), and flash memory devices; magnetic disks, for example, internal hard disks or removable disks; magneto-optical disks; and CD-ROM, DVD- R, DVD-RAM, and DVD-ROM disks. The memory may store various objects or data, including caches, classes, frameworks, applications, backup data, jobs, web pages, web page templates, database tables, repositories storing business and dynamic information, and any other appropriate information including any parameters, variables, algorithms, instructions, rules, constraints, or references. Additionally, the memory may include any other appropriate data, such as logs, policies, security or access data, reporting files, as well as others. The processor and the memory can be supplemented by, or incorporated in, special purpose logic circuitry.

To provide for interaction with a user, implementations of the subject matter described in this specification can be implemented on a computer having a display device, for example, a cathode ray tube (CRT), liquid crystal display (LCD), light emitting diode (LED), or plasma monitor, for displaying information to the user and a keyboard and a pointing device, for example, a mouse, trackball, or trackpad by which the user can provide input to the computer. Input may also be provided to the computer using a touchscreen, such as a tablet computer surface with pressure sensitivity, a multi-touch screen using capacitive or electric sensing, or other type of touchscreen. Other kinds of devices can be used to provide for interaction with a user as well; for example, feedback provided to the user can be any form of sensory feedback, for example, visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

The term “graphical user interface,” or “GUI,” may be used in the singular or the plural to describe one or more graphical user interfaces and each of the displays of a particular graphical user interface. Therefore, a GUI may represent any graphical user interface, including but not limited to, a web browser, a touch screen, or a command line interface (CLI) that processes information and efficiently presents the information results to the user. In general, a GUI may include multiple user interface (UI) elements, some or all associated with a web browser, such as interactive fields, pull-down lists, and buttons operable by the business suite user. These and other UI elements may be related to or represent the functions of the web browser.

Implementations of the subject matter described in this specification can be implemented in a computing system that includes a back-end component, for example, as a data server, or that includes a middleware component, for example, an application server, or that includes a front-end component, for example, a client computer having a graphical user interface or a web browser through which a user can interact with an implementation of the subject matter described in this specification, or any combination of one or more such back-end, middleware, or front-end components. The components of the system can be interconnected by any form or medium of wireline or wireless digital data communication, for example, a communication network. Examples of communication networks include a local area network (LAN), a radio access network (RAN), a metropolitan area network (MAN), a wide area network (WAN), worldwide interoperability for microwave access (WIMAX), a wireless local area network (WLAN) using, for example, 902.11 a/b/g/n and 902.20, all or a portion of the Internet, and any other communication system or systems at one or more locations. The network may communicate with, for example, internet protocol (IP) packets, frame relay frames, asynchronous transfer mode (ATM) cells, voice, video, data, or other suitable information between network addresses.

The computing system can include clients and servers. A client and server are generally remote from each other and typically interact through a communication network. The relationship of client and server arises by virtue of computer programs running on the respective computers and having a client-server relationship to each other.

In some implementations, any or all of the components of the computing system, both hardware and software, may interface with each other or the interface using an application programming interface (API) or a service layer. The API may include specifications for routines, data structures, and object classes. The API may be either computer language-independent or -dependent and refer to a complete interface, a single function, or even a set of APIs. The service layer provides software services to the computing system. The functionality of the various components of the computing system may be accessible for all service consumers via this service layer. Software services provide reusable, defined business functionalities through a defined interface. For example, the interface may be software written in any suitable language providing data in any suitable format. The API and service layer may be an integral or a stand-alone component in relation to other components of the computing system. Moreover, any or all parts of the service layer may be implemented as child or sub-modules of another software module, enterprise application, or hardware module without departing from the scope of this specification.

While this specification contains many specific implementation details, these should not be construed as limitations on the scope of any invention or on the scope of what may be claimed, but rather as descriptions of features that may be specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described as acting in certain combinations and even initially claimed as such, one or more features from a claimed combination can in some cases be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing may be advantageous and performed as deemed appropriate.

Moreover, the separation or integration of various system modules and components in the implementations described earlier should not be understood as requiring such separation or integration in all implementations, and it should be understood that the described program components and systems can generally be integrated together in a single software product or packaged into multiple software products.

Accordingly, the earlier provided description of example implementations does not define or constrain this specification. Other changes, substitutions, and alterations are also possible without departing from the spirit and scope of this specification.

What is claimed is:

1. A well tracer detection system comprising:
a tracer detection system in fluid communication with a water line of a Gas Oil Separation Plant (GOSP) via a fluid sampling line fluidically connected to the water line, wherein the GOSP is configured to:
receive commingled well hydrocarbon fluids from a plurality of wells, each of the plurality of wells in communication with a corresponding subterranean zone of a hydrocarbon reservoir, the corresponding subterranean zone tagged with a respective tracer, the respective tracer flowing into the well in response to a well breakthrough,
separate the commingled well hydrocarbon fluids into hydrocarbon components comprising water, and
flow the water through the water line,
wherein the tracer detection system is configured to:
receive a fluid sample from the fluid sampling line,
analyze the fluid sample to determine a presence of the tracers with which the subterranean zones corresponding to the plurality of wells were tagged,
identify, in response to analyzing the fluid sample, a tracer in the fluid sample, and
identify, in response to identifying the tracer in the fluid sample, a well corresponding to a subterranean zone tagged with the identified tracer.

2. The system of claim 1, wherein the tracer detection system is configured to detect tracers in a part per trillion (ppt) or part per quadrillion (ppq) range.

3. The system of claim 1, wherein the tracer detection system comprises at least one of a laser-driven fluorescent spectrometer or a gas chromatography mass spectrometer.

4. The system of claim 3, wherein each tracer comprises at least one of an optically-tagged nanoparticle tracer or a mass-tagged nanoparticle tracer.

5. The system of claim 1, further comprising a fluid valve fluidically connecting the water line and the fluid sampling line, the fluid valve configured to actuate to transfer the fluid sample from the water line to the fluid sampling line.

6. The system of claim 1, further comprising a filter positioned between the fluid sampling line and the tracer detection system, the filter configured to remove contaminants in the fluid sample.

7. The system of claim 6, wherein the contaminants exclude a tracer.

8. The system of claim 1, further comprising a pump configured to flow the fluid sample from the fluid sampling line to the tracer detection system.

9. The system of claim 1, further comprising a computer system comprising:
one or more processors; and
a computer-readable medium storing:
a plurality of first identifiers identifying the corresponding plurality of wells and a plurality of second identifiers identifying the tracers with which the subterranean zones corresponding to the plurality of wells are tagged, and
computer instructions executable by the one or more processors to perform operations, wherein, to identify the well tagged with the identified tracer, the computer system is configured to:
identify, from among the plurality of second identifiers, a second identifier identifying the tracer identified by the tracer detection system, and
identify, from among the plurality of first identifiers, a first identifier identifying the well corresponding to the subterranean zone tagged with the identified tracer.

10. The system of claim 1, further comprising a transmitter configured to transmit an identity of the identified well to a control station.

11. The system of claim 1, wherein a volume of the fluid sample is substantially 50 milliliters or less.

12. A method of identifying a well breakthrough, comprising:
flowing a fluid sample from a water line of a Gas Oil Separation Plant (GOSP) to a tracer detection system, wherein the GOSP is configured to:
receive commingled well hydrocarbon fluids from a plurality of wells, each of the plurality of wells in communication with a corresponding subterranean zone of a hydrocarbon reservoir, the corresponding subterranean zone tagged with a respective tracer, the respective tracer flowing into the well in response to a well breakthrough,
separate the commingled well hydrocarbon fluids into hydrocarbon components comprising water, and
flow the water through the water line;
analyzing, by the tracer detection system, the fluid sample for a presence of the tracers in the fluid sample;
identifying, by the tracer detection system, a subterranean zone from the subterranean zones tagged with the tracers; and
identifying, by the tracer detection system, a well corresponding to the identified subterranean zone.

13. The method of claim 12, wherein flowing the fluid sample to the tracer detection system comprises pumping, using a pump, the fluid sample to the tracer detection system.

14. The method of claim 12, wherein flowing the fluid sample to the tracer detection system comprises gravitationally flowing the fluid sample to the tracer detection system.

15. The method of claim 12, further comprising:
receiving an identity of the identified well; and
monitoring, at the identified well in the hydrocarbon reservoir, a flow of the identified tracer into the identified well.

16. The method of claim 12, wherein determining the tracer in the fluid sample comprises detecting the tracer by performing at least one of laser-driven fluorescent spectroscopy or gas chromatography mass spectroscopy on the fluid sample.

17. The method of claim 16, wherein a concentration of the tracer in the fluid sample is in a part per trillion (ppt) or part per quadrillion (ppq) range.

18. The method of claim 16, wherein each tracer comprises at least one of an optically-tagged nanoparticle tracer or a mass-tagged nanoparticle tracer.

19. The method of claim 12, wherein identifying the well of the plurality of wells with which the determined tracer was tagged comprises:
identifying a first computer-stored identifier that identifies the tracer; and
identifying a second computer-stored identifier that is mapped to the identified first computer-stored identifier, the second computer-stored identifier identifying the well.

20. The method of claim 19, wherein identifying the first computer-stored identifier and the second computer-stored identifier comprises:
storing, in a computer-readable storage medium, a plurality of first identifiers identifying the tracers and a plurality of second identifiers identifying the plurality of wells, the plurality of first identifiers comprising the first computer-stored identifier, the plurality of second identifiers comprising the second computer-stored identifier; and
searching, by one or more processors operatively coupled to the computer-readable storage medium, the plurality of first identifiers and the plurality of second identifiers for a first identifier that identifies the tracer and a second identifier mapped to the first identifier.

21. The method of claim 12, further comprising actuating a fluid valve fluidically connecting the water line and a fluid sampling line to transfer the fluid sample from the water line to the fluid sampling line that is fluidically connected to the tracer detection system.

22. The method of claim 12, further comprising filtering contaminants from the fluid sample before flowing the fluid sample to the tracer detection system.

23. The method of claim 22, wherein the contaminants exclude a tracer.

24. A hydrocarbon reservoir monitoring method comprising:
periodically sampling fluid carried by a water line of a Gas Oil Separation Plant (GOSP), wherein the GOSP is configured to receive commingled well hydrocarbon fluids from a plurality of wells formed in a plurality of regions of a hydrocarbon reservoir, separate the commingled well hydrocarbon fluids into hydrocarbon components comprising water, and flow the water through the water line, wherein the plurality of regions are tagged with a plurality of tracers, each tracer injected into a respective region of the hydrocarbon reservoir surrounding a respective well, wherein each tracer flows from the respective region into the respective well in response to a well breakthrough;
analyzing each sampled fluid for one or more tracers of the plurality of tracers; and
monitoring the plurality of wells for fluid breakthrough based on results of analyzing each sampled fluid.

25. The method of claim 24, wherein periodically sampling the fluid comprises sampling the fluid once or twice in a day.

26. The method of claim 24, wherein monitoring the plurality of wells for fluid breakthrough based on results of analyzing each sampled fluid comprises:
determining, in response to analyzing a fluid sample, a presence of a tracer in the fluid sample;
identifying a region of the hydrocarbon reservoir surrounding a well into which the tracer was injected; and
monitoring the well for fluid breakthrough.
